(19) United States
(12) Patent Application Publication
(10) Pub. No.: US 2025/0025426 A1
(43) Pub. Date: Jan. 23, 2025

(54) SYNERGISTIC TRANSPORT OF LIPOPHILIC AND HYDROPHILIC ACTIVE SUBSTANCES IN NANOPARTICLES

(71) Applicants: KARLSRUHER INSTITUT FÜR TECHNOLOGIE, Karlsruhe (DE); MAX-PLANCK-GESELLSCHAFT ZUR FORDERUNG DER WISSENSCHAFTEN E.V., München (DE); GEORG-AUGUST-UNIVERSITÄT STIFTUNG ÖFFENTLICHEN RECHTS, UNIVERSITÄTSMEDIZIN, Göttingen (DE)

(72) Inventors: David RUDOLPH, Keltern/Niebelsbach (DE); Claus FELDMANN, Ettlingen (DE); Joanna NAPP, Rosdorf (DE); Frauke ALVES, Göttingen (DE)

(21) Appl. No.: 18/715,394

(22) PCT Filed: Oct. 14, 2022
(86) PCT No.: PCT/EP2022/078646

§ 371 Date: May 31, 2024

(30) Foreign Application Priority Data
Dec. 6, 2021 (EP) 21212525.6

(51) Int. Cl. A61K 9/51; A61K 31/407; A61K 31/4164; A61K 31/438; A61K 31/4439; A61K 31/496; A61K 31/498; A61K 31/5383; A61K 31/65; A61K 31/7052; A61K 49/00; A61K 51/12

(52) U.S. Cl. CPC A61K 9/5115; A61K 31/407; A61K 31/4164; A61K 31/438; A61K 31/4439; A61K 31/496; A61K 31/498; A61K 31/5383; A61K 31/65; A61K 31/7052; A61K 49/0002; A61K 51/1251; A61K 2123/00

(57) ABSTRACT

The present invention relates to nanocontainers for the synergistic transport of lipophilic and hydrophilic active ingredients or detection reagents. In particular, the nanocontainers according to the invention offer a possibility for diagnosing and/or treating diseases with combinations of active ingredients (therapy) and detection reagents (diagnostics), which have different solubility properties. The present invention further relates to a method for producing the nanocontainers according to the invention.

The present invention relates to nanocontainers for the synergistic transport of lipophilic and hydrophilic active ingredients or detection reagents. In particular, the nanocontainers according to the invention offer a possibility for diagnosing and/or treating diseases with combinations of active ingredients (therapy) and detection reagents (diagnostics), which can have different solubility properties. The present invention further relates to a method for producing the nanocontainers according to the invention.

Lipophilic compounds, in particular pharmaceutically active ingredients, are often excluded from effective clinical use because they cannot be administered at all or only with great difficulty and/or only reach the site of action in insufficient concentrations. This is particularly the case when lipophilic compounds are administered intravenously via the bloodstream or when lipophilic compounds are to be introduced into an aqueous milieu (e.g. intravenous or intraperitoneal administration). In addition, cell uptake or transport through membranes is often significantly reduced for lipophilic compounds compared to hydrophilic compounds.

Here, nanocontainers or nanoparticles offer a well-known platform for the transport of pharmaceutically active ingredients, such as chemotherapeutic drugs for the treatment of tumor diseases. The nanocontainers support the direct and protected delivery of medication into the tumor and can thus improve the effectiveness of the chemotherapeutic drug and/or avoid possible side effects. In oncology, these include non-PEGylated liposomal doxorubicin (Myocet®) or PEGylated liposomal doxorubicin (Caelyx®), which show improved cardiotoxicity, neutropenia and/or alopecia compared to the free active ingredient. Furthermore, PEGylated liposomal irinotecan (Onivyde®) or nanoparticulate albumin-bound paclitaxel (Abraxane®) enable the use of highly hydrophobic and highly potent taxanes, which can be administered in higher doses, in a shorter time, and without co-medication.

Disadvantages of liposomal nanocontainers or nanoparticles include inter alia their short half-life and stability in suspension. Furthermore, they are expensive to produce and sterilization is only possible to a limited extent due to their sensitivity to high temperatures and types of radiation.

Therefore, further nanoparticle-based concepts for the transport of chemotherapeutic drugs have been proposed by materials scientists. These concepts are based on organic matrices, for example polymers or biopolymers, or inorganic matrices, for example silicon dioxide, iron oxide or metal phosphates, in which the active pharmaceutical ingredient is embedded.

A disadvantage here are toxic components difficult to break down under physiological conditions, which can lead to the occurrence of significant side effects. A long-term carcinogenic effect has been shown for silicon dioxide in particular.

Furthermore, the active ingredient is only superficially bound to or into the organic or inorganic matrix. In addition, the amount of active ingredient based on the total mass of the nanoparticles with the matrix as the majority component is usually small (<20%). Lipophilic active ingredients also show poor stability in suspension, which results in the active ingredient being released too quickly or too slowly. Furthermore, such matrices offer only limited options for transporting lipophilic, pharmaceutically active ingredients and for combining different active ingredients. This is why, despite a complex material system, such systems are usually only used for one clinical condition. For this reason, studies with organic and inorganic matrices for the transport of pharmaceutically active ingredients have so far been limited in most cases to in vitro experiments.

There is therefore a need for new concepts that offer a cost-effective and efficient way to provide lipophilic compounds, for example pharmaceutically active ingredients or detection reagents, as a combination preparation with hydrophilic compounds, for example pharmaceutically active ingredients or detection reagents, in order to achieve lower dosages and shorter treatment times and avoid side effects.

The object described above is achieved by the embodiments of the present invention characterized in the claims.

In particular, according to the invention, a nanocontainer is provided, comprising a lipophilic core,
wherein the lipophilic core comprises at least one lipophilic compound selected from a lipophilic, pharmaceutically active ingredient or a lipophilic detection reagent; and a hydrophilic casing or shell enclosing the lipophilic core,
wherein the hydrophilic shell is composed of an inorganic-organic hybrid compound as an ionic compound, wherein the inorganic-organic hybrid compound is composed of an inorganic metal cation selected from Mn²⁺, Sc³⁺, Y³⁺, La³⁺, Fe²⁺, Fe³⁺, [ZrO]²⁺, [HfO]²⁺, Bi³⁺, Gd³⁺ or a lanthanide Ln²⁺ or Ln³⁺ or a hydrated form of these cations (e.g. [Gd(H₂O)ₙ]³⁺ with n=2-8, [Gd(OH)]²⁺, [GdO]⁺), and a water-soluble organic active ingredient anion or a water-soluble detection reagent anion, each of which contains at least one phosphate, phosphonate, sulfate, sulfonate, carbonate or carboxylate group as a functional group.

As described above, the lipophilic core of the nanocontainer according to the invention is formed or constructed from at least one lipophilic compound, the lipophilic compound not being subject to any particular limitation. According to the invention, the term “lipophilic compound” is understood to mean compounds that are substantially hardly soluble in water (<0.1 mol L−1) and easily soluble (>1.0 mol L−1) in alkanes, for example hexane or dodecane and/or aromatic hydrocarbons, for example toluene. The lipophilic core can comprise either one or more lipophilic compounds.

According to the present invention, the at least one lipophilic compound is selected from a lipophilic, pharmaceutically active ingredient or a lipophilic detection reagent. According to the invention, the term “pharmaceutically active ingredient” is understood to mean a substance that is used as an agent for curing or preventing human or animal diseases, as well as a substance that is intended to be used in or on the human or animal body to restore, improve or influence human or animal body functions. According to the invention, the term “detection reagent” is understood to mean a substance or a compound that can be detected/localized in the body after administration, for example optically via fluorescence in the case of a fluorescent dye or also through X-ray absorption, magnetic measurements or based on their radioactive radiation. In particular, the term “detection reagent” is not understood to mean surface-active agents (“surfactants”) such as monododecyl phosphate. Under certain circumstances, a compound can be both an active ingredient and a detection reagent. For example, the chemotherapeutic drug irinotecan itself shows blue fluorescence, the cytostatics of the anthracycline group usually show fluorescence, for example doxorubicin fluoresces red, and the antiviral dolutegravir shows red fluorescence.

According to the present invention, the lipophilic core is enclosed by a shell based on an inorganic-organic hybrid compound. This structure stabilizes the lipophilic core and makes it available as a transport and storage form.

The nanocontainers according to the invention do not contain any polymers or polymer compounds that make up the core or shell.

The shell of the nanocontainer according to the invention comprises or consists or is made up of at least one inorganic-organic hybrid compound, which, as an ionic compound, in turn is made up of an inorganic metal cation and a water-soluble, organic anion, which is an organic active ingredient anion and/or a hydrophilic detection reagent anion.

According to the present invention, the inorganic metal cation of the hydrophilic shell is selected from the group consisting of Mn²⁺, Sc³⁺, Y³⁺, La³⁺, Fe²⁺, Fe³⁺, [ZrO]²⁺, [HfO]²⁺, Bi³⁺, Gd³⁺ or a lanthanide Ln²⁺ or Ln³⁺ or a hydrated form of these cations (e.g. [Gd(H₂O)ₙ]³⁺ with n=2-8, [Gd(OH)]²⁺, [GdO]⁺), or mixtures thereof. Particularly preferably, the inorganic cation is selected from the group consisting of Gd³⁺, [Gd(OH)]²⁺, [GdO]⁺, and [ZrO]²⁺.

By selecting certain metal cations, selected from the list defined above, the nanocontainer can be provided with additional properties. In particular, by selecting heavy, magnetic and/or radioactive inorganic metal cations, for example [ZrO]²⁺, [⁸⁹ZrO]²⁺, ²²⁵Ac³⁺, [HfO]²⁺, Bi³⁺, Gd³⁺ oder Mn²⁺, the nanocontainers can be detected by X-ray absorption, magnetic measurements and/or by radioactive decay.

According to the present invention, the inorganic-organic hybrid compound is an ionic compound comprising a hydrophilic organic active ingredient anion or a hydrophilic detection reagent anion. The definitions of the “active ingredient anion” and the “detection reagent anion” correspond to the above-mentioned definitions regarding the “pharmaceutically active ingredient” and the “detection reagent”.

Furthermore, the water-soluble organic active ingredient anion or the water-soluble detection reagent anion each contains at least one phosphate, phosphonate, sulfate, sulfonate, carbonate or carboxylate group as a functional group in order to form the inorganic-organic hybrid compound as an ionic compound together with the inorganic metal cation, which ionic compound forms the shell of the nanocontainers according to the invention that encloses the lipophilic core. The inorganic-organic hybrid compound itself is hardly soluble in water.

According to a preferred embodiment of the present invention, the at least one lipophilic, pharmaceutically active ingredient is selected from
the group of antibiotics consisting of delamanid, bedaquiline, benzothiazinones such as benzothiazinon 043, clofazimine, rifampicin, levofloxacin, cefaclor, cefpodoxime, imipenem, meropenem, ciprofloxacin, levofloxacin, norfloxacin, chloramphenicol, trimethoprim, azithromycin, metronidazole, linezolid, tyrothricin, rifabutin, rifaximin, fusidic acid, doxycycline, hydroxytamoxifen, and pantoprazole; or
the group of antiviral drugs consisting of amantadine, rimantadine, penciclovir, emivirine, FGI-106, maraviroc, sofosbuvir, baloxavirmarboxil, etravirine, nevirapine, atazanavir, indinavir, lopinavir, nelfinavir, tipranavir, boceprevir, telaprevir, dolutegravir, raltegravir, and tecovirimat; or
the group of chemotherapeutic drugs consisting of ifosfamide, paclitaxel, docetaxel, abraxan, taxoter, mechlorethamine, erlotinib, gefitinib, imatinib, vemurafenib, cisplatin, daunorubicin, epirubicin, lomustine, vismodegib, actinomycin D, vinorelbine, camptothecin, topotecan, irinotecan, etoposide, teniposide, and mercaptopurine; or
the group of anti-inflammatory drugs consisting of cannabidiol, triamcinolone, budesonide, diclofenac, cortisol, calcitriol, leflunomide, and non-steroidal anti-inflammatory drugs.

Furthermore, according to a further preferred embodiment of the present invention, the detection reagent is selected from the group of fluorescent dyes consisting of Lumogen Red, Lumogen Orange, Lumogen Yellow or Lumogen Green, magnesium phthalocyanine, zinc phthalocyanine, 1,1′-diethyl-4,4′-carbocyanine iodide, 3,3′-diethylthiadicarbocyanine iodide, magnesium tetraphenylporphyrin, and phthalocyanine.

According to a preferred embodiment of the present invention, the mass of the lipophilic, pharmaceutically active ingredient and/or the lipophilic detection reagent is 50 to 100% by weight, based on the total mass of the lipophilic core, preferably at least 60% by weight, particularly preferably at least 70% by weight, and most preferably at least 75% by weight. Since the carrier system of the nanocontainer, i.e. the other components other than the active ingredient, usually does not have a pharmaceutical effect, a large loading amount of active ingredient can be achieved with the nanocontainer according to the invention, so that a very high pharmaceutical effectiveness per amount of nanocontainer administered can be achieved.

According to an embodiment of the present invention, the mass of the lipophilic, pharmaceutically active ingredient is less than 100% by weight if the lipophilic core comprises one or more lipophilic excipients.

“Lipophilic excipients” are understood to mean compounds that have a beneficial effect on the properties of the lipophilic core. An example of this is α-tocopherol, which has antioxidant properties and can therefore lead to improved stability. Other lipophilic excipients may be toluene, phellandrene or natural oils such as oleic acid or linolenic acid.

According to an embodiment of the present invention, the lipophilic core has a diameter of 10 to 150 nm, measured by electron microscopy. The lipophilic core preferably has a diameter of at least 10 nm, more preferably at least 20 nm, and most preferably at least 30 nm. A diameter below the minimum mentioned above is technically difficult to achieve.

More preferably, the lipophilic core has a diameter of preferably at most 120 nm, more preferably at most 80 nm, and most preferably at most 50 nm.

As stated above, the shell of the nanocontainer is formed from an inorganic-organic hybrid compound. In addition to the metal cation, this inorganic-organic hybrid compound is made up of a hydrophilic organic active ingredient anion or a hydrophilic detection reagent anion.

According to a preferred embodiment of the present invention, the hydrophilic active ingredient anion or the hydrophilic detection reagent anion is selected from the group of antibiotics consisting of clindamycin phosphate, erythromycin phosphate, tedizolid phosphate, CpG oligodeoxynucleotides, fosfomycin, moxalactam, ceftriaxone, amoxicillin, phenoxymethylpenicillin, aztreonam, moxifloxacin, and bacitracin; or
the group of antiviral drugs consisting of idoxuridin phosphate, acyclovir phosphat, penciclovir phosphate, ganciclovir phosphate, remdesivir phosphat, galidesivir phosphat, vidarabin phosphate, ribavirin phosphate, abacavir phosphate, stavudine phosphate, adefovir, fosamprenavir, fostemsavir, tenofovir, brincidofovir, cidofovir, foscarnet, ivermectin, bevirimat, and zanamivir, or
the group of chemotherapeutic drugs consisting of 5-fluoro-2′-deoxyuridine-5′-monophosphate, gemcitabine monophosphate, gemcitabine triphosphate, fludarabine, pemetrexed, methotrexate, estramustine phosphate, streptozotocin phosphate, mitoxantrone phosphate, azacitidine phosphate, cyclophosphamide mustard, SN-38, melphalan, chlorambucil, and bendamustine; or
the group of anti-inflammatory drugs consisting of betamethasone phosphate, dexamethasone phosphate, prednisolone phosphate, sulfasalazine, acetyl salicylate, methotrexate, ibuprofen, naproxen, and ketoprofen; or
the group of fluorescent dyes consisting of phenylumbelliferone phosphate, flavin mononucleotide, methylfluorescein phosphate, resorufin phosphate, Dynomics-546-uridine triphosphate, Dynomics-647-uridine triphosphate, Amaranth Red, Chicago Sky Blue, Direct Blue 71, Congo Red, Nuclear Fast Red, Acid Red 97, and Evans Blue.

As far as the compounds listed above are concerned, they are present as anions. The commonly used active ingredient names are used here, i.e. clindamycin phosphate (although the starting compound is not the anion, but the acid or the sodium salt), ibuprofen (correct as the starting compound, however, as the active ingredient in the nanoparticles the anion is present), etc. It is known to the person skilled in the art that the corresponding anion forms or can be produced by dissolving the acid or the sodium salt in water.

In the case of SN-38, which itself does not have any of the functional groups defined according to the invention, the aqueous solution thereof must be made alkaline so that the inner cyclic ester opens and a free carboxyl function is created.

According to an embodiment of the present invention, the mass of the hydrophilic, pharmaceutically active ingredient and/or the detection reagent is 50 to 90% by weight, based on the total mass of organic anions in the shell, preferably at least 60% by weight, particularly preferably at least 70% by weight, and most preferably at least 75% by weight. Since the carrier system of the nanocontainer, i.e. the other components other than the active ingredient, usually does not have a pharmaceutical effect, a large loading amount of active ingredient can be achieved with the nanocontainer according to the invention, so that a very high pharmaceutical effectiveness per amount of nanocontainer administered can be achieved.

If, for example, a lipophilic, pharmaceutically active ingredient is used in the core and a (hydrophilic) detection reagent is used in the casing or shell, the nanocontainer according to the invention can advantageously release the active ingredient after administration and can be localized by the detection reagent in e.g. cells, tissues, and organs.

According to a further embodiment of the present invention, the nanocontainer has a diameter of 20 to 300 nm, measured by electron microscopy. The nanocontainer particularly preferably has a diameter of 30 nm or more and most preferably of 50 nm or more. If the diameter of the nanocontainers is above the lower limit mentioned above, the nanocontainer has advantageous stability and a sufficient amount of active ingredient per nanocontainer.

More preferably, the nanocontainer has a diameter of 250 nm or less, more preferably of 150 nm or less, and most preferably of 100 nm or less.

A further aspect of the present invention relates to a method for producing such nanocontainers. The core-shell particles are substantially produced using the solvent-antisolvent method (see FIG. 2). Here, a highly concentrated, preferably saturated solution of the lipophilic active ingredient or detection reagent is first prepared in a suitable solvent. This solvent solution is injected into a polar antisolvent as quickly as possible with intensive stirring and/or intensive ultrasonic mixing. It is preferably water or a mixture of water with other water-miscible solvents. The prerequisite is that the active ingredient dissolves very well in the solvent but very poorly in the antisolvent. The solvent must still be miscible with the antisolvent at least within a certain concentration range. The injection of the solvent solution into the antisolvent then leads to the precipitation of the lipophilic active ingredient or detection reagent with the formation of nanoparticles. Subsequently, the inorganic-organic hybrid compound-based shell is deposited on the nanoparticles consisting of the lipophilic active ingredient or detection reagent (see FIG. 2). For this purpose, the organic functional anion is usually added first. The solution containing the inorganic cation is then slowly added dropwise, whereby the inorganic-organic hybrid compound is slowly deposited on the lipophilic particle core and enclosed it. Optionally, intermediate layers (e.g. surfactants such as tocopherol phosphate or monododecyl phosphate) can be used to increase and improve the adhesion and deposition of hydrophilic hybrid compound on the lipophilic particle core.

All of the above statements regarding the nanocontainer according to the invention also apply to the method according to the invention for producing the nanocontainer.

In step (I) of the method according to the invention, a solution of the at least one lipophilic compound is provided, the lipophilic compound being a lipophilic, pharmaceutically active ingredient and/or a lipophilic detection reagent. For example, methanol, ethanol, 1-propanol, 2-propanol, butanol, tetrahydrofuran, dioxane, benzyl alcohol, dimethyl sulfoxide, acetonitrile, dimethylformamide, acetone, hexane, dodecane, toluene or mixtures thereof can be used as a solvent. Preferred solvents are ethanol, benzyl alcohol, acetone, tetrahydrofuran, and dimethyl sulfoxide.

According to a further embodiment, the solution of the at least one lipophilic compound can further contain a lipophilic excipient, for example tocopherol phosphate or monodecyl phosphate. The lipophilic excipient can increase the stability of the lipophilic core. If no lipophilic excipient is added, the loading amount of lipophilic compound per nanocontainer increases significantly.

In step (II) of the method according to the invention, the solution provided in step (I) is then injected into a polar solvent. For example, deionized water, aqueous NaCl solutions, ethanol, dimethyl sulfoxide or mixtures thereof can be used as polar solvents. Preferred solvents are deionized water or aqueous NaCl solutions.

According to a further embodiment of the present invention, the lipophilic excipient does not have to be contained in the solution provided in step (I). Optionally, one or more lipophilic excipients can also be provided in the polar solvent.

Furthermore, one or more chemical compounds, for example ionic compounds, can be contained in the polar solvent. Ammonium acetate, which stabilizes the pH value of the polar solvent, can be mentioned here as an example.

In step (III), the organic anion and the inorganic cation, which form the inorganic-organic hybrid compound to build up the shell, are then added one after the other, usually in this order.

The reaction temperature of the method according to the invention is not subject to any particular limitation. Typically a temperature range between −50° C. and +90° C. is used. Cooling with ice or dry ice or a suitable cooling liquid (e.g. cooled with a cryostat) can be useful to reduce the solubility of the substances. Preferably, the method is carried out at room temperature.

After carrying out step (III), the nanocontainer according to the invention formed usually precipitates or is present in suspension in the solvent used. After step (III), step (IV) can optionally be carried out. This optional step (IV) includes isolating and/or purifying the precipitated nanocontainer. Such isolation and/or purification is not limited and can be carried out by any suitable method. Such methods are known in the prior art.

Isolation and/or purification of the nanocontainers preferably takes place by a method selected from the group consisting of centrifugation techniques, dialysis techniques, phase transfer techniques, chromatography techniques, washing techniques, and combinations thereof. The methods mentioned above can also be combined and/or carried out multiple times.

A further aspect of the present invention relates to a nanoparticle comprising the nanocontainer according to the invention, functionalized with at least one element selected from the group consisting of antibodies, peptides, 5-aminolevulinic acid, folic acid derivatives, albumin derivatives, saccharides and ligands, for specific binding to receptors of cells. Thereby, targeted transport of the nanocontainer and targeted release of the active ingredients or detection reagents is achieved.

A further aspect of the present invention relates to the use of the nanocontainer according to the invention in the treatment of infections caused by bacteria and/or viruses, inflammatory autoimmune reactions or for the treatment of tumors.

The figures show:

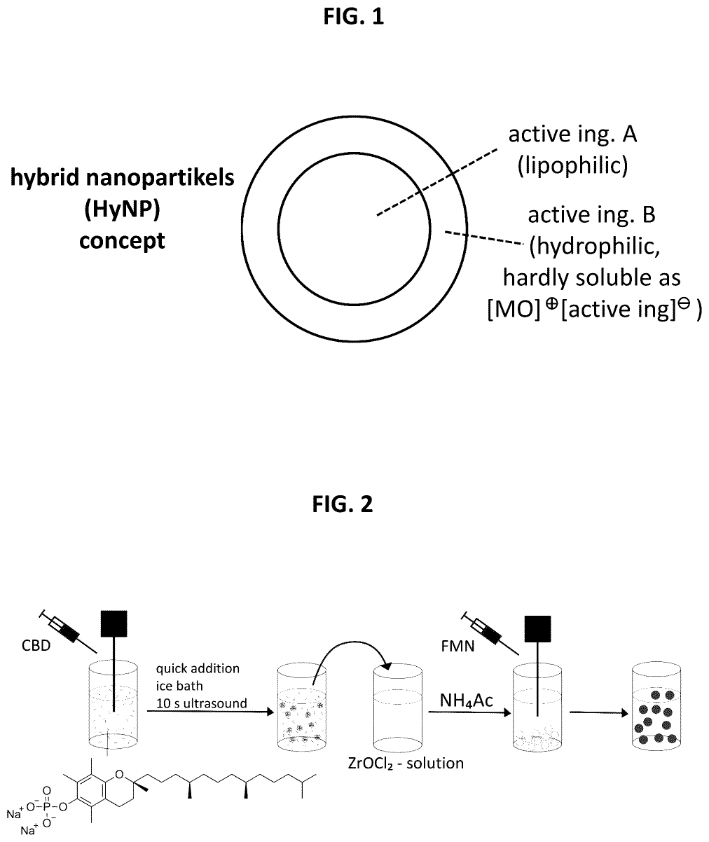

FIG. 1 shows the structure according to the invention of the core@shell nanocontainers with a lipophilic active ingredient as the core and an inorganic-organic hybrid compound as the shell.

FIG. 2 shows the synthesis according to the invention of core@shell nanocontainers with the formation of the lipophilic core and the hydrophilic shell using the example of CBD@[ZrO]²⁺[FMN]²⁻, where CBD: cannabidiol forms the core and FMN: flavin mononuclide forms the shell.

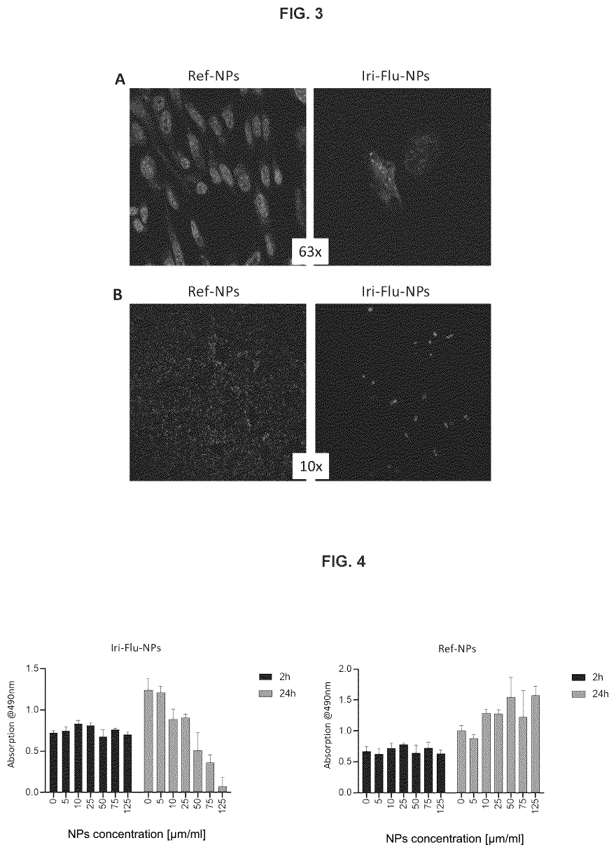

FIG. 3 shows microscopy images according to embodiment 2 below, which shows the internalization of the core@shell nanocontainers by pancreatic tumor cells. 25,000 cells (13,000 cells cm⁻²) were plated on coverslips. After 24 h, the cells were treated with the ITC@[ZrO]²⁺[FLU]²⁻-core@shell nanocontainers and the reference nanocontainers (50 μL/500 μL medium) for 48 h, counterstained with DAPI (nuclei staining), fixed and analyzed under a confocal fluorescence microscope. (A) shows a close-up view under 63× magnification; the nanocontainers (the DUT-546 dye) are shown in the light areas, the dark areas show the nuclei stained with DAPI. (B) shows an overview image (10× magnification) showing nuclei stained with DAPI; the toxic effect of the ITC@[ZrO]²⁺[FLU]²⁻-core@shell nanocontainers (hardly any cells present) compared to the reference nanocontainers (a confluent cell carpet) is clear.

FIG. 4 shows diagrams according to embodiment 3 below, in which 10,000 breast cancer cells (pH8N8) are plated with a concentration of 30,000 cells cm⁻² and treated with increasing concentrations of the ITC@[ZrO]²⁺[FLU]²⁻-core@shell nanocontainers and the reference nanocontainers, which do not contain any pharmaceutically active ingredient. Cell growth inhibition efficacy was measured using the CellTiter 96@AQueous One Solution Cell Proliferation Assay before (2 h) and after treatment (24 h). The experiment was carried out in triplicate, the bars represent the mean values, and the error bars represent the standard deviation.

### EXAMPLES

The following examples serve to illustrate the present invention, but are not limited thereto.

### Embodiment 1.1: Production of CBD@[ZrO]²⁺[FMN]²⁻-Nanocontainers

Tocopherol phosphate (disodium salt; 3.8 mg, 7.0 μmol) and ammonium acetate (36.1 mg, 468 μmol) are dissolved in 6 mL of deionized water each. Both solutions are combined and cooled with an ice bath. A solution of cannabidiol (CBD; 2.3 mg, 7.2 μmol) in 0.2 mL ethanol is injected with intensive stirring. During and after the injection, the solution/suspension is additionally mixed using a 10 s long ultrasound pulse. The resulting suspension with CBD nanoparticles, which represent the future nanocontainer core, is colloidally stable for about 1 h.

In the next step, the suspension of the CBD nanoparticles is added dropwise to 9 mL of a ZrOCl₂ solution (octahydrate; 6.8 mg, 37.9 μmol) over 2 min with intensive stirring. This suspension is stirred for a further 10 minutes. The mixture is then centrifuged (10 min, 13,000 rpm). The nanoparticles are then resuspended in 6 mL of the ammonium acetate solution described above using an ultrasonic wand (1 min) or by intensive stirring. After 30 s, 5 mL of a flavin mononucleotide solution (FMN, disodium salt; 3.2 mg, 7.0 μmol) is injected over a period of 10 s and stirred for a further 10 min. In this way, [ZrO]²⁺[FMN]²⁻ as an inorganic-organic hybrid compound is deposited as a shell on the CBD core, so that CBD@[ZrO]²⁺[FMN]²⁻-core@shell nanocontainers are formed. The resulting yellow suspension is centrifuged (10 min, 25000 rpm), and the CBD@[ZrO]²⁺[FMN]²⁻-core@shell nanocontainers are resuspended in deionized water.

The CBD@[ZrO]²⁺[FMN]²⁻-core@shell nanocontainers with anti-inflammatory active ingredients and fluorescent dye are colloidally very stable as a suspension in water. According to electron microscopy, they have an average diameter of 50 nm, with the core having a diameter of about 20 nm and the shell having a thickness of approximately 15 nm.

### Embodiment 1.2: Production of ERB@[ZrO]²⁺[FdUMP]²⁻-Nanocontainers

Tocopherol phosphate (disodium salt; 3.8 mg, 7.0 μmol) and ammonium acetate (36.1 mg, 468 μmol) are dissolved in 6 mL of deionized water each. Both solutions are combined and cooled with an ice bath. A solution of epirubicin (ERB; 3.9 mg, 7.2 μmol) in 0.2 mL ethanol is injected with intensive stirring. During and after the injection, the solution/suspension is additionally mixed using a 10 s long ultrasound pulse. The resulting suspension with ERB nanoparticles, which represent the future nanocontainer core, is colloidally stable for about 1 h.

In the next step, the suspension of the ERB nanoparticles is added dropwise to 9 mL of a ZrOCl₂ solution (octahydrate; 6.8 mg, 37.9 μmol) over 2 min with intensive stirring. This suspension is stirred for a further 10 minutes. The mixture is then centrifuged (10 min, 13,000 rpm). The nanoparticles are then resuspended in 6 mL of the ammonium acetate solution described above using an ultrasonic wand (1 min) or by intensive stirring. After 30 s, 5 mL of a 5-fluoro-2′-deoxyuridine-5′-monophosphate solution (FdUMP, disodium salt; 2.3 mg, 7.0 μmol) is injected over a period of 10 s and stirred for a further 10 min. In this way, [ZrO]²⁺[FdUMP]²⁻ as an inorganic-organic hybrid compound is deposited as a shell on the ERB core, so that ERB@[ZrO]²⁺[FdUMP]²⁻-core@shell nanocontainers are formed. The resulting suspension is centrifuged (10 min, 25000 rpm), and the ERB@[ZrO]²⁺[FdUMP]²⁻-core@shell nanocontainers are resuspended in deionized water.

The ERB@[ZrO]²⁺[FdUMP]²⁻-core@shell nanocontainers with two chemotherapeutic active ingredients are colloidally very stable as a suspension in water. According to electron microscopy, they have an average diameter of about 60 nm, with the core having an average diameter of about 30 nm and the shell having a thickness of about 15 nm.

By adding Lumogen Red to the ERB solution (1 mol % based on the amount of ERB) and/or adding Dynomics 647 uridine triphosphate to the FdUMP solution (0.1 mol % based on the amount of FdUMP) nanocontainer core and/or nanocontainer shell can be fluorescently marked. The person skilled in the art is aware of the absorption and emission behavior of the respective fluorescent dyes.

### Embodiment 1.3: Production of PAC@[Gd(OH)]²⁺[GemP]²⁻-Nanocontainers

Tocopherol phosphate (disodium salt; 3.8 mg, 7.0 μmol) and ammonium acetate (36.1 mg, 468 μmol) are dissolved in 6 mL of deionized water each. Both solutions are combined and cooled with an ice bath. A solution of paclitaxel (PAC; 6.1 mg, 7.2 μmol) in 0.2 mL tetrahydrofuran is injected with intensive stirring. During and after the injection, the solution/suspension is additionally mixed using a 10 s long ultrasound pulse. The resulting suspension with PAC nanoparticles, which represent the future nanocontainer core, is colloidally stable for about 1 h.

In the next step, the suspension of the PAC nanoparticles is added dropwise to 9 mL of a GdCl₃ solution (hexahydrate; 14.1 mg, 37.9 μmol) over 2 min with intensive stirring. This suspension is stirred for a further 10 minutes. The mixture is then centrifuged (10 min, 13,000 rpm). The nanoparticles are then resuspended in 6 mL of the ammonium acetate solution described above using an ultrasonic wand (1 min) or by intensive stirring. After 30 s, 5 mL of a gemcitabine monophosphate solution (GemP, disodium salt; 2.4 mg, 7.0 μmol) is injected over a period of 10 s and stirred for a further 10 min. In this way, [Gd(OH)]²⁺[GemP]²⁻ as an inorganic-organic hybrid compound is deposited as a shell on the PAC core, so that PAC@[Gd(OH)]²⁺[GemP]²⁻-core@shell nanocontainers are formed. The resulting suspension is centrifuged (10 min, 25000 rpm), and the PAC@[Gd(OH)]²⁺[GemP]²⁻-core@shell nanocontainers are resuspended in deionized water.

The PAC@[Gd(OH)]²⁺[GemP]²⁻-core@shell nanocontainers with two chemotherapeutic active ingredients are colloidally very stable as a suspension in water. According to electron microscopy, they have an average diameter of about 50 nm, with the core having an average diameter of about 20 nm and the shell having a thickness of about 15 nm.

By adding Lumogen Green to the PAC solution (1 mol % based on the amount of PAC) and/or adding Dynomics 647 uridine triphosphate to the GemP solution (0.1 mol % based on the amount of GemP), nanocontainer core and/or nanocontainer shell can be fluorescently marked. The person skilled in the art is aware of the absorption and emission behavior of the respective fluorescent dyes.

### Embodiment 1.4: Production of AMT@[ZrO]²⁺[RemP]²⁻-Nanocontainers

Tocopherol phosphate (disodium salt; 3.8 mg, 7.0 μmol) and ammonium acetate (36.1 mg, 468 μmol) are dissolved in 6 mL of deionized water each. Both solutions are combined and cooled with an ice bath. A solution of amantadine (AMT; 1.1 mg, 7.2 μmol) in 0.4 mL tetrahydrofuran is injected with intensive stirring. During and after the injection, the solution/suspension is additionally mixed using a 10 s long ultrasound pulse. The resulting suspension with AMT nanoparticles, which represent the future nanocontainer core, is colloidally stable for about 1 h.

In the next step, the suspension of the AMT nanoparticles is added dropwise to 9 mL of a ZrOCl₂ solution (octahydrate; 6.8 mg, 37.9 μmol) over 2 min with intensive stirring. This suspension is stirred for a further 10 minutes. The mixture is then centrifuged (10 min, 13,000 rpm). The nanoparticles are then resuspended in 6 mL of the ammonium acetate solution described above using an ultrasonic wand (1 min) or by intensive stirring. After 30 s, 5 mL of a remdesivir phosphate solution (RemP, disodium salt; 2.6 mg, 7.0 μmol) is injected over a period of 10 s and stirred for a further 10 min. In this way, [ZrO]²⁺[RemP]²⁻ as an inorganic-organic hybrid compound is deposited as a shell on the AMT core, so that AMT@[ZrO]²⁺[RemP]²⁻-core@shell nanocontainers are formed. The resulting suspension is centrifuged (10 min, 25,000 rpm), and the AMT@[ZrO]²⁺[RemP]²⁻-core@shell nanocontainers are resuspended in deionized water.

The AMT@[ZrO]²⁺[RemP]²⁻-core@shell nanocontainers with two antiviral active ingredients are colloidally very stable as a suspension in water. According to electron microscopy, they have an average diameter of about 40 nm, with the core having an average diameter of about 20 nm and the shell having a thickness of about 10 nm.

By adding Lumogen Orange to the AMT solution (1 mol % based on the amount of AMT) and/or adding Dynomics 546 uridine triphosphate to the RemP solution (0.1 mol % based on the amount of RemP), nanocontainer core and/or nanocontainer shell can be fluorescently marked. The person skilled in the art is aware of the absorption and emission behavior of the respective fluorescent dyes.

### Embodiment 1.5: Production of ERB@[ZrO]²⁺[FdUMP]²⁻-Nanocontainers

Tocopherol phosphate (disodium salt; 1.9 mg, 3.5 μmol) and ammonium acetate (36.1 mg, 468 μmol) are dissolved in 6 mL of deionized water each. Both solutions are combined and cooled with an ice bath. A solution of epirubicin (ERB; 7.8 mg, 14.4 μmol) in 0.4 mL ethanol is injected with intensive stirring. During and after the injection, the solution/suspension is additionally mixed using a 10 s long ultrasound pulse. The resulting suspension with ERB nanoparticles, which represent the future nanocontainer core, is colloidally stable for about 1 h.

In the next step, the suspension of the ERB nanoparticles is added dropwise to 9 mL of a ZrOCl₂ solution (octahydrate; 6.8 mg, 37.9 μmol) over 2 min with intensive stirring. This suspension is stirred for a further 10 minutes. The mixture is then centrifuged (10 min, 13,000 rpm). The nanoparticles are then resuspended in 6 mL of the ammonium acetate solution described above using an ultrasonic wand (1 min) or by intensive stirring. After 30 s, 5 mL of a 5-fluoro-2′-deoxyuridine-5′-monophosphate solution (FdUMP, disodium salt; 4.5 mg, 14.0 μmol) is injected over a period of 10 s and stirred for a further 10 min. In this way, [ZrO]²⁺[FdUMP]²⁻ as an inorganic-organic hybrid compound is deposited as a shell on the ERB core, so that ERB@[ZrO]²⁺[FdUMP]²⁻-core@shell nanocontainers are formed. The resulting suspension is centrifuged (10 min, 25000 rpm), and the ERB@[ZrO]²⁺[FdUMP]²⁻-core@shell nanocontainers are resuspended in deionized water.

The ERB@[ZrO]²⁺[FdUMP]²⁻-core@shell nanocontainers with two chemotherapeutic active ingredients are colloidally very stable as a suspension in water. According to electron microscopy, they have an average diameter of about 80 nm, with the core having an average diameter of about 40 nm and the shell having a thickness of about 20 nm.

By adding Lumogen Red to the ERB solution (1 mol % based on the amount of ERB) and/or adding Dynomics 647 uridine triphosphate to the FdUMP solution (0.1 mol % based on the amount of FdUMP), nanocontainer core and/or nanocontainer shell can be fluorescently marked. The person skilled in the art is aware of the absorption and emission behavior of the respective fluorescent dyes.

### Embodiment 1.6: Production of DCF@[Gd(OH)]²⁺[BMP]²⁻-Nanocontainers

Tocopherol phosphate (disodium salt; 3.8 mg, 7.0 μmol) and ammonium acetate (36.1 mg, 468 μmol) are dissolved in 6 mL of deionized water each. Both solutions are combined and cooled with an ice bath. A solution of diclofenac (DCF, sodium salt; 2.3 mg, 7.2 μmol) in 0.2 mL of benzyl alcohol is injected with intensive stirring. During and after the injection, the solution/suspension is additionally mixed using a 10 s long ultrasound pulse. The resulting suspension with DCF nanoparticles, which represent the future nanocontainer core, is colloidally stable for about 1 h.

In the next step, the suspension of the DCF nanoparticles is added dropwise to 9 mL of a GdCl₃ solution (hexahydrate; 14.1 mg, 37.9 μmol) over 2 min with intensive stirring. This suspension is stirred for a further 10 minutes. The mixture is then centrifuged (10 min, 13,000 rpm). The nanoparticles are then resuspended in 6 mL of the ammonium acetate solution described above using an ultrasonic wand (1 min) or by intensive stirring. After 30 s, 5 mL of a betamethasone phosphate solution (BMP, disodium salt; 3.6 mg, 7.0 μmol) is injected over a period of 10 s and stirred for a further 10 min. In this way, [Gd(OH)]²⁺[BMP]²⁻ as an inorganic-organic hybrid compound is deposited as a shell on the DCF core, so that DCF@[Gd(OH)]²⁺[BMP]²⁻-core@shell nanocontainers are formed. The resulting suspension is centrifuged (10 min, 25000 rpm), and the DCF@[Gd(OH)]²⁺[BMP]²⁻-core@shell nanocontainers are resuspended in deionized water.

The DCF@[Gd(OH)]²⁺[BMP]²⁻-core@shell nanocontainers with two anti-inflammatory active ingredients are colloidally very stable as a suspension in water. According to electron microscopy, they have an average diameter of about 50 nm, with the core having an average diameter of about 20 nm and the shell having a thickness of about 15 nm.

By adding 3,3-diethylthiadicarbocyanine iodide to the DCF solution (1 mol % based on the amount of DCF) and/or adding flavin mononuclide to the BMP solution (5.0 mol % based on the amount of BMP), nanocontainer core and/or nanocontainer shell can be fluorescently marked. The person skilled in the art is aware of the absorption and emission behavior of the respective fluorescent dyes.

### Embodiment 1.7: Production of BDQ@[ZrO]²⁺[CLP]²⁻-Nanocontainers

Tocopherol phosphate (disodium salt; 3.8 mg, 7.0 μmol) and ammonium acetate (36.1 mg, 468 μmol) are dissolved in 6 mL of deionized water each. Both solutions are combined and cooled with an ice bath. A solution of bedaquiline (BDQ; 4.0 mg, 7.2 μmol) in 0.3 mL of dimethyl sulfoxide is injected with intensive stirring. During and after the injection, the solution/suspension is additionally mixed using a 10 s long ultrasound pulse. The resulting suspension with BDQ nanoparticles, which represent the future nanocontainer core, is colloidally stable for about 1 h.

In the next step, the suspension of the BDQ nanoparticles is added dropwise to 9 mL of a ZrOCl₂ solution (octahydrate; 6.8 mg, 37.9 μmol) over 2 min with intensive stirring. This suspension is stirred for a further 10 minutes. The mixture is then centrifuged (10 min, 13,000 rpm). The nanoparticles are then resuspended in 6 mL of the ammonium acetate solution described above using an ultrasonic wand (1 min) or by intensive stirring. After 30 s, 5 mL of a clindamycin phosphate solution (CLP, disodium salt; 3.8 mg, 7.0 μmol) is injected over a period of 10 s and stirred for a further 10 min. In this way, [ZrO]²⁺[CLP]²⁻ as an inorganic-organic hybrid compound is deposited as a shell on the BDQ core, so that BDQ@[ZrO]²⁺[CLP]²⁻-core@shell nanocontainers are formed. The resulting suspension is centrifuged (10 min, 25000 rpm), and the BDQ@[ZrO]²⁺[CLP]²⁻-core@shell nanocontainers are resuspended in deionized water.

The BDQ@[ZrO]²⁺[CLP]²⁻-core@shell nanocontainers with two antibiotic active ingredients are colloidally very stable as a suspension in water. According to electron microscopy, they have an average diameter of about 40 nm, with the core having an average diameter of about 20 nm and the shell having a thickness of about 10 nm.

By adding 3,3′-diethylthiadicarbocyanine iodide to the BDQ solution (1 mol % based on the amount of BDQ) and/or adding Dynomics 647 uridine triphosphate to the CLP solution (0.1 mol % based on the amount on CLP), nanocontainer core and/or nanocontainer shell can be fluorescently marked. The person skilled in the art is aware of the absorption and emission behavior of the respective fluorescent dyes.

### Embodiment 1.8: Production of ITC@[ZrO]²⁺[FLU]²⁻-Nanocontainers

Tocopherol phosphate (disodium salt; 3.8 mg, 7.0 μmol) and ammonium acetate (36.1 mg; 468 μmol) are dissolved in 6 mL of deionized water each. Both solutions are combined and cooled with an ice bath. A solution of irinotecan (ITC; 4.2 mg, 7.2 μmol) in 0.2 mL of benzyl alcohol is injected with intensive stirring. During and after the injection, the solution/suspension is additionally mixed using a 10 s long ultrasound pulse. The resulting suspension with ITC nanoparticles, which represent the future nanocontainer core, is colloidally stable for about 1 h.

In the next step, the suspension of the ITC nanoparticles is added dropwise to 9 mL of a ZrOCl₂ solution (octahydrate; 6.8 mg, 37.9 μmol) over 2 min with intensive stirring. This suspension is stirred for a further 10 minutes. The mixture is then centrifuged (10 min, 13,000 rpm). The nanoparticles are then resuspended in 6 mL of the ammonium acetate solution described above using an ultrasonic wand (1 min) or by intensive stirring. After 30 s, 5 mL of a fludarabine solution (FLU, disodium salt; 2.9 mg, 7.0 μmol) is injected over a period of 10 s and stirred for a further 10 min. In this way, [ZrO]²⁺[FLU]²⁻ as an inorganic-organic hybrid compound is deposited as a shell on the ITC core, so that ITC@[ZrO]²⁺[FLU]²⁻-core@shell nanocontainers are formed. The resulting suspension is centrifuged (10 min, 25000 rpm), and the ITC@[ZrO]²⁺[FLU]²⁻-core@shell nanocontainers are resuspended in deionized water.

The ITC@[ZrO]²⁺[FLU]²⁻-core@shell nanocontainers with two chemotherapeutic active ingredients are colloidally very stable as a suspension in water. According to electron microscopy, they have an average diameter of about 60 nm, with the core having an average diameter of about 30 nm and the shell having a thickness of about 15 nm.

By adding Lumogen Red to the ITC solution (1 mol % based on the amount of ITC) and/or adding Dynomics 546 uridine triphosphate to the FLU solution (0.1 mol % based on the amount of FLU), nanocontainer core and/or nanocontainer shell are fluorescently marked. The person skilled in the art is aware of the absorption and emission behavior of the respective fluorescent dyes.

### Embodiment 1.9: Production of PAC@[GdO]⁺[SN-38]⁻-Nanocontainers

Tocopherol phosphate (disodium salt, 3.8 mg, 7.0 μmol) and ammonium acetate (36.1 mg, 468 μmol) are dissolved in 6 mL of deionized water each. Both solutions are combined and cooled with an ice bath. A solution of paclitaxel (PAC; 6.1 mg, 7.2 μmol) in 0.2 mL DMSO is injected with intensive stirring. During and after the injection, the solution/suspension is additionally mixed using a 10 s long ultrasound pulse. The resulting suspension with PAC nanoparticles, which represent the future particle core, is colloidally stable for about 1 h.

In the next step, the suspension of the PAC nanoparticles is added dropwise to 9 mL of a GdCl₃ solution (hexahydrate, 14.1 mg, 37.9 μmol) over 2 min with intensive stirring. This suspension is stirred for a further 10 minutes. The mixture is then centrifuged (10 min, 13,000 rpm). The nanoparticles are then resuspended in 6 mL of deionized water using an ultrasonic wand (1 min) or by intensive stirring. After 30 s, 5 mL of an alkaline SN-38 solution (SN-38 as the active metabolite of irinotecan, pH 9, 2.7 mg, 7.0 μmol) is injected over a period of 10 s and stirred for a further 10 min. In this way, [GdO]⁺[SN-38]⁻ as an inorganic-organic hybrid compound is deposited as a shell on the PAC core to form PAC@[GdO]⁺[SN-38]⁻-core@shell nanoparticles. The resulting suspension is centrifuged (25,000 rpm, 10 min), and the PAC@[GdO]⁺[SN-38]⁻-core@shell nanoparticles are resuspended in deionized water.

The PAC@[GdO]⁺[SN-38]⁻-core@shell nanoparticles with two chemotherapeutic active ingredients are colloidally very stable as a suspension in water. According to electron microscopy, they have an average diameter of about 50 nm, with the core having an average diameter of about 20 nm and the shell having a thickness of about 15 nm.

By adding Lumogen Green to the PAC solution (1 mol % based on the amount of PAC) and/or adding Dynomics 647 uridine triphosphate to the SN-38 solution (0.1 mol % based on the amount of SN-38), particle core and/or particle shell can be fluorescently marked. The person skilled in the art is aware of the absorption and emission behavior of the respective fluorescent dyes.

### Embodiment 2: Uptake of Core@-Shell Nanocontainers

Embodiment 2 relates to the evaluation of the uptake of the core@shell nanocontainers by pancreatic tumor cells. ITC@[ZrO]²⁺[FLU]²⁻-core@shell nanocontainers, as well as corresponding reference nanocontainers not containing any pharmaceutically active ingredient, were compared.

FIG. 3A shows a greatly enlarged uptake/internalization of the particles after 48 h of incubation of the core@shell nanocontainers with the tumor cells (Panc02 cell line). As expected, the ITC@[ZrO]²⁺[FLU]²⁻-core@shell nanocontainers resulted in severe toxicity, resulting in only a few cells being present in the preparation. This can be seen even more clearly in FIG. 3B (overview image, 10× magnification).

### Embodiment 3: Effectiveness of the Core@-Shell Nanocontainers

Embodiment 3 shows the effectiveness of the core@shell nanocontainers on murine mammary carcinoma cells. The measurement was carried out using a CellTiter 96@AQueous One Solution Cell Proliferation Assay (MTS), which is a colorimetric method for determining cell viability.

As shown in FIG. 4, treatment of the tumor cells with ITC@[ZrO]²⁺[FLU]²⁻-core@shell nanocontainers leads to a concentration-dependent toxicity, while incubation with the reference nanocontainers not containing any pharmaceutically active ingredient does not show any negative influence on cell growth.

1. A nanocontainer, comprising
a lipophilic core,
wherein the lipophilic core comprises at least one lipophilic compound selected from a lipophilic, pharmaceutically active ingredient or a lipophilic detection reagent; and
a hydrophilic shell enclosing the lipophilic core,
wherein the hydrophilic shell is composed of an inorganic-organic hybrid compound as an ionic compound, wherein the inorganic-organic hybrid compound is composed of an inorganic metal cation selected from Mn²⁺, Sc³⁺, Y³⁺, La³⁺, Fe²⁺, Fe³⁺, [ZrO]²⁺, [HfO]²⁺, Bi³⁺, Gd³⁺ or a lanthanide Ln²⁺ or Ln³⁺ or a hydrated form of these cations, and an organic active ingredient anion or a hydrophilic detection reagent anion, each of which contains at least one phosphate, phosphonate, sulfate, sulfonate, carbonate or carboxylate group as a functional group,
wherein a pharmaceutically active ingredient is a substance used as an agent for curing or preventing human or animal diseases, as well as a substance intended to be used in or on the human or animal body to restore, improve or influence human or animal body functions, and a detection reagent is a substance that can be detected/localized in the body after administration, for example optically via fluorescence in the case of a fluorescent dye or also through X-ray absorption, magnetic measurements or based on their radioactive radiation.

2. The nanocontainer according to claim 1, wherein the at least one lipophilic, pharmaceutically active ingredient or lipophilic detection reagent is selected from the group of antibiotics consisting of delamanid, bedaquiline, benzothiazinones such as benzothiazinon 043, clofazimine, rifampicin, levofloxacin, cefaclor, cefpodoxime, imipenem, meropenem, ciprofloxacin, levofloxacin, norfloxacin, chloramphenicol, trimethoprim, azithromycin, metronidazole, linezolid, tyrothricin, rifabutin, rifaximin, fusidic acid, doxycycline, hydroxytamoxifen, and pantoprazole; or
the group of antiviral drugs consisting of amantadine, rimantadine, penciclovir, emivirine, FGI-106, maraviroc, sofosbuvir, baloxavirmarboxil, etravirine, nevirapine, atazanavir, indinavir, lopinavir, nelfinavir, tipranavir, boceprevir, telaprevir, dolutegravir, raltegravir, and tecovirimat; or
the group of chemotherapeutic drugs consisting of ifosfamide, paclitaxel, docetaxel, abraxan, taxoter, mechlorethamine, erlotinib, gefitinib, imatinib, vemurafenib, cisplatin, daunorubicin, epirubicin, lomustine, vismodegib, actinomycin D, vinorelbine, camptothecin, topotecan, irinotecan, etoposide, teniposide, and mercaptopurine; or
the group of anti-inflammatory drugs consisting of cannabidiol, triamcinolone, budesonide, diclofenac, cortisol, calcitriol, leflunomide, and non-steroidal anti-inflammatory drugs; or
the group of fluorescent dyes consisting of Lumogen Red, Lumogen Orange, Lumogen Yellow or Lumogen Green, magnesium phthalocyanine, zinc phthalocyanine, 1,1′-diethyl-4,4′-carbocyanine iodide, 3,3′-diethylthiadicarbocyanine iodide, magnesium tetraphenylporphyrin, and phthalocyanine.

3. The nanocontainer according to claim 1, wherein the mass of the lipophilic, pharmaceutically active ingredient and/or the lipophilic detection reagent is 50 to 100% by weight, based on the total mass of the lipophilic core.

4. The nanocontainer according to claim 1, wherein the lipophilic core further comprises a lipophilic excipient.

5. The nanocontainer according to claim 1, wherein the lipophilic core has a diameter of 10 to 150 nm, measured by electron microscopy.

6. The nanocontainer according to claim 1, wherein the hydrophilic active ingredient anion or the hydrophilic detection reagent anion in the inorganic-organic hybrid compound is selected from
the group of antibiotics consisting of clindamycin phosphate, erythromycin phosphate, tedizolid phosphate, CpG oligodeoxynucleotides, fosfomycin, moxalactam, ceftriaxone, amoxicillin, phenoxymethylpenicillin, aztreonam, moxifloxacin, and bacitracin; or
the group of antiviral drugs consisting of idoxuridin phosphate, acyclovir phosphat, penciclovir phosphate, ganciclovir phosphate, remdesivir phosphat, galidesivir phosphat, vidarabin phosphate, ribavirin phosphate, abacavir phosphate, stavudine phosphate, adefovir, fosamprenavir, fostemsavir, tenofovir, brincidofovir, cidofovir, foscamet, ivermectin, bevirimat, and zanamivir, or
the group of chemotherapeutic drugs consisting of 5-fluoro-2′-deoxyuridine-5′-monophosphate, gemcitabine monophosphate, gemcitabine triphosphate, fludarabine, pemetrexed, methotrexate, estramustine phosphate, streptozotocin phosphate, mitoxantrone phosphate, azacitidine phosphate, cyclophosphamide mustard, SN-38, melphalan, chlorambucil, and bendamustine; or
the group of anti-inflammatory drugs consisting of betamethasone phosphate, dexamethasone phosphate, prednisolone phosphate, sulfasalazine, acetyl salicylate, methotrexate, ibuprofen, naproxen, and ketoprofen; or
the group of fluorescent dyes consisting of phenylumbelliferone phosphate, flavin mononucleotide, methylfluorescein phosphate, resorufin phosphate, Dynomics-546-uridine triphosphate, Dynomics-647-uridine triphosphate, Amaranth Red, Chicago Sky Blue, Direct Blue 71, Congo Red, Nuclear Fast Red, Acid Red 97, and Evans Blue.

7. The nanocontainer according to claim 1, wherein the mass of the hydrophilic, pharmaceutically active ingredient and/or the detection reagent is 50 to 90% by weight, based on the total mass of organic anions in the shell,

8. The nanocontainer according to claim 1, wherein the nanocontainer has a diameter of 20 to 300 nm, measured by electron microscopy.

9. The nanocontainer according to claim 1, wherein the at least one lipophilic compound is a lipophilic, pharmaceutically active ingredient.

10. The nanocontainer according to claim 1, wherein the inorganic-organic hybrid compound is composed of an inorganic metal cation as defined above and an organic active ingredient anion to form the hydrophilic shell.

11. A method for producing a nanocontainers according to claim 1 by means of solvent-antisolvent method, comprising the steps of
(I) providing a solution of the at least one lipophilic compound, wherein the at least one lipophilic compound is a lipophilic, pharmaceutically active ingredient and/or a lipophilic detection reagent;
(II) injecting the solution provided in step (I) into a polar solvent; and
(III) adding the organic anion and the inorganic cation which form the inorganic-organic hybrid compound to form the hydrophilic shell.

12. A nanoparticle, comprising:
the nanocontainer according to claim 1, functionalized with at least one element selected from the group consisting of antibodies, peptides, 5-aminolevulinic acid, folic acid derivatives, albumin derivatives, saccharides and ligands, for specific binding to receptors of cells.

13. A nanocontainer according to 1 and/or a nanoparticle according to claim 12 for use in the treatment of infections caused by bacteria and/or viruses, inflammatory autoimmune reactions or for the treatment of tumors.
